(19) United States
(12) Patent Application Publication
(10) Pub. No.: US 2025/0017575 A1
(43) Pub. Date: Jan. 16, 2025

(54) Biopsy Devices, Methods, and Systems

(71) Applicant: James E. SELIS, Birmingham, MI (US)

(72) Inventor: James E. SELIS, Birmingham, MI (US)

(21) Appl. No.: 18/834,886

(22) PCT Filed: Feb. 10, 2023
(86) PCT No.: PCT/US2023/012765

§ 371 Date: Jul. 31, 2024

Related U.S. Application Data
(60) Provisional application No. 63/480,761, filed on Jan. 20, 2023.
(60) Provisional application No. 63/308,634, filed on Feb. 10, 2022.

(51) Int. Cl. A61B 10/02; A61B 90/00

(52) U.S. Cl. CPC A61B 10/0275; A61B 2090/3966; A61B 2090/3987

(57) ABSTRACT

Methods, devices, systems, and kits for implementing a technique for introducing a marker into a biopsy cavity of a patient in need of a biopsy. The marker is introduced by a biopsy probe assembly, which includes a biopsy needle having a longitudinal axis and an opening facing away from the longitudinal axis and through which a biopsy sample can be taken at a biopsy site. The marker is delivered to the biopsy site through the opening with a biopsy marker deployment device. At least a portion of the biopsy marker deployment device is rotated or is caused to rotate about its longitudinal axis to bar re-entry of the marker into the biopsy needle through the opening. A unique encapsulated marker is disclosed that can be used with the aforementioned method or independent of it.

### RELATED APPLICATIONS

This application claims priority to and incorporates by reference in their entirety U.S. Provisional Application Nos. 63/308,634 (filed Feb. 10, 2022) and 63/480,761 (filed Jan. 20, 2023).

### FIELD

The present teachings relate in general to biopsy devices, methods, and systems and more particularly to devices, methods, and systems for precision location of a marker while a probe is inserted.

### BACKGROUND

After performing a biopsy (e.g., a breast biopsy), it is common to insert a marker into the breast (i.e., a breast of a patient in need of a biopsy) in a region adjoining or including the biopsy site, desirably directly within a biopsy cavity (i.e., the space previously occupied by the lesion removed by the biopsy). This helps in subsequent monitoring, as the marker is typically detectable radiologically. For example, it is common in percutaneous needle breast biopsy for a marker containing a radiopaque material, usually metal, to be inserted within a biopsy cavity, or at the region adjoining the cavity (e.g., desirably less than 1 cm away from the cavity). The marker is visible on mammography. Some of the markers are also visible by way of ultrasonography, magnetic resonance imaging (“MRI”), or both.

The purpose of placing the marker is for future reference. So, if the biopsy proves benign, then when the patient has future mammograms, the radiologist will know that the lesion has been previously biopsied. If the lesion proves to be high risk or malignant then the marker can provide a target for surgical resection. Sometimes, at the time of a biopsy (e.g., a needle biopsy), all of the abnormality is removed. In these cases, the biopsy marker is the only target for resection. For this reason, accurate placement of the marker has come to be regarded as important.

Unfortunately, even using the most sophisticated of instruments commercially available today according to their instructed usage, inaccurate deployment occurs frequently, and by some estimations as high as about 30% of all marker deployments.

Without intending to be bound by theory, one possible cause of the inaccuracies is due to an incomplete deployment of a marker into the biopsy cavity. It is believed that this is potentially may be occasioned by forces from the inherent elasticity of breast tissue that is compressed during the biopsy procedure. Upon deployment of the marker, the elasticity exerts a force against the marker as it is being deployed. In some instances, this may have an effect of causing the marker to be forced back into the opening of a biopsy instrument. In turn, this could result in the marker being dragged backward upon withdrawal of the instrument. Though staying within the breast, as a result of such dragging, the marker may not be present near the biopsy cavity. For instance, the marker may end up found in tissue remote from the biopsy cavity, e.g., 2 cm or further away from the cavity.

Once located within a biopsy site, one concern of the marker is the geometry thereof puncturing or otherwise damaging tissue surrounding the biopsy site. Another concern is the bioactivity of the marker as it remains within the patient over time.

Accordingly, there is a need for an improved method of marker placement into a biopsy cavity generally, and in particular an improved method of marker placement into a biopsy cavity (e.g., a cavity resulting from a percutaneous needle breast biopsy). There is also a need for improved devices and systems for marker placement into a breast biopsy cavity (e.g., a cavity resulting from a percutaneous needle breast biopsy).

### SUMMARY

The present teachings envision in one general sense a method, devices employed in the method, and a system for implementing the method, for introducing a tissue marker device into a biopsy cavity. The method may comprise the steps of: providing a biopsy probe assembly including a probe body and a biopsy needle connected to the probe body, the biopsy needle including a longitudinal axis and an opening facing away from the longitudinal axis and through which a tissue sample can be taken; inserting the biopsy needle into a patient in need of a biopsy to position it at a biopsy site; removing a volume of the tissue sample from the patient, while the biopsy needle is inserted into the patient, at the biopsy site thereby defining a biopsy cavity; delivering the tissue marker device to the biopsy site through the opening with a biopsy marker deployment device; and rotating or causing at least a portion of the biopsy marker deployment device and/or the biopsy needle to rotate about its longitudinal axis to bar re-entry of the tissue marker device into the biopsy needle through the opening.

The present teachings overcome the above problem by providing a unique method, device, kit, and system. Among the benefits of the present teachings is that a tissue marker device can be deployed to a biopsy site (e.g., within a biopsy cavity) and it will remain at or near the site (e.g., no greater than 10 millimeters (mm), 7 mm, or 5 mm away from a biopsy site after deployment). The precise location of a tissue marker device makes possible its future detection at the biopsy site during radiographic examination (e.g., at least one year, two years, three years, or even longer, after a biopsy).

One unique aspect of the teachings involves a manipulation of a manipulable biopsy probe assembly. Specifically, while keeping the biopsy probe (and its associated components) at a predetermined location within a patient in need of a biopsy (and from which a biopsy tissue same has been taken), a tissue marker device may be delivered to a biopsy site, e.g., within a biopsy cavity. The tissue marker device may be delivered to a location at least partially, or fully within the biopsy cavity. The tissue marker device may be delivered through an opening (e.g., a side opening) of each of a biopsy marker deployment device and a biopsy needle of the biopsy probe. The opening may face sideways relative to the longitudinal axes, respectively, of the biopsy marker deployment device and the biopsy needle. The biopsy marker deployment device may include a driver surrounded by a sheath having a longitudinal axis. The driver may be employed to deliver the tissue marker device.

After the tissue marker device has exited the opening of the biopsy needle, the biopsy marker deployment device (in particular, at least a sheath of the biopsy marker deployment device) is rotated or caused to rotate about its longitudinal axis to bar re-entry of the marker into the probe assembly. For instance, the rotation is a sufficient amount (e.g., at least +/−, 120°, 150°, ideally 180° or 540° (or additional like increments (e.g., 270°, 360° or otherwise) of the previously recited amounts)), so that the opening (e.g., the side opening in a sheath of the biopsy marker deployment device) faces away from the biopsy cavity. The biopsy needle may be maintained at the biopsy site before and/or during said rotation. The biopsy marker deployment device may be rotated within no more than 20 seconds, preferably 15 seconds, more preferably 10 seconds, still more preferably 5 seconds, or still more preferably 3 seconds following the tissue marker device (including any encasement) exiting the biopsy marker deployment device, the biopsy needle, or both.

A wall portion of the biopsy marker deployment device (particularly, a wall portion of a sheath of the biopsy marker deployment device) may thus be in direct facing relation with the marker to prevent re-entry into the opening. As will be appreciated, the re-entry can be prevented as a result of the biopsy marker deployment device (e.g., the wall portion, specifically the wall portion of the biopsy marker deployment device sheath) blocking a path toward re-entry.

After this, the biopsy needle likewise may be rotated about its longitudinal axis. In this manner, a wall portion of the biopsy needle may be positioned in direct facing relation with the marker. The result of the rotation of the biopsy needle may be that a wall portion of the biopsy marker deployment device (e.g., a sheath wall portion) faces the opening of the biopsy needle, the opening of the biopsy marker deployment device faces an internal surface of a wall portion of the biopsy needle, or both.

The above method may be automated or at least partially automated using devices or systems described herein. In this regard, the biopsy marker deployment device may be rotated or caused to rotate manually, automatically with aid of a motor, or both. The biopsy marker deployment device may be rotated or caused to rotate upon detecting (e.g., with a sensor) an exit of the tissue marker device (including any encasement) from the biopsy marker deployment device, the biopsy needle, or both. Upon detecting an exit of a marker from the biopsy marker deployment device, the biopsy needle, or both, a signal may be transmitted to an electronic controller programmed to actuate a motor for causing the rotation of at least the sheath of the biopsy marker deployment device.

It is contemplated that a cutter may be employed for the step of removing a volume of tissue sample from the patient. The cutter may be coaxially disposed within the biopsy needle.

The device may include a biopsy probe assembly configured for use in the above method. The biopsy probe assembly may include a probe body and a biopsy needle connected to the probe body, the biopsy needle including a longitudinal axis and an opening facing away (e.g., sideways) from the longitudinal axis and through which a biopsy sample can be taken; and a marker deployment device configured to advance a tissue marker device through the side opening of the biopsy needle; and a motor that is adapted to be automatically actuated upon a tissue marker device exiting the opening of the biopsy needle to rotate the biopsy marker deployment device about its longitudinal axis to bar re-entry of the marker into the biopsy needle through the opening.

A length of the opening along the longitudinal axis may be generally equal to or less than a length of the tissue marker device (including any encasement and/or branches thereof) along a longitudinal axis thereof. The length of the tissue marker device may be about 1 or more, 1.2 or more, 1.5 or more, 2 or more, or even 3 or more times the length of the opening. A width of the opening along an axis transverse to the longitudinal axis may be generally equal to a width of the tissue marker device along an axis transverse to the longitudinal axis thereof.

The biopsy marker deployment device and/or the biopsy needle may comprise a ramp. The ramp may be within the distal tip portion (e.g., of a sheath) of the biopsy marker deployment device. The ramp may be proximate a distal tip of the biopsy needle. The ramp may be juxtaposed with the opening. The ramp may be oriented at an angle of about 10 or more, 15 or more, 20 or more, 25 or more, 30 or more, 35 or more, 40 or more, or even 45 or more degrees relative to the longitudinal axis of the biopsy needle and/or the biopsy marker device. The ramp may be oriented at an angle of about 75 or less, 70 or less, 65 or less, 60 or less, or even 55 or less degrees relative to the longitudinal axis of the biopsy needle and/or the biopsy marker device.

The biopsy marker deployment device may comprise one or more stops that prevent further depression of the driver. One or more stops may cause a distal tip of the driver to be spaced about 1 mm or more, 2 mm or more, 3 mm or more, or even 4 mm or more from the distal tip of the biopsy needle when the driver is fully depressed relative to the biopsy needle. One or more stops may cause a distal tip of the driver to be spaced about 15 mm or less, 14 mm or less, 13 mm or less, 12 mm or less, 11 mm or less, or even 10 mm or less from the distal tip of the biopsy needle when the driver is fully depressed relative to the biopsy needle.

The length of the driver along the longitudinal axis thereof may be about 0.95 or less, 0.9 or less, 0.85 or less, or even 0.8 or less times the length of the biopsy needle along the longitudinal axis thereof. The length of the driver along the longitudinal axis thereof may be about 0.65 or more, 0.7 or more, or even 0.75 or more times the length of the biopsy needle along the longitudinal axis thereof.

The system may include the biopsy probe assembly described above and may be configured for use in the above method. The system may comprise an electronic controller in signaling combination with the motor, and a sensor that detects an exit of a tissue marker device from the opening of the biopsy marker deployment device, the biopsy needle, or both, wherein upon the sensor detecting the exit, it issues a sensor signal to the electronic controller, and in response to the sensor signal the electronic controller issues an actuation signal to the electric motor to cause the rotation of at least a portion of the biopsy marker deployment device.

The present teachings envision in one general sense a tissue marker device configured for deployment into a patient in need of a biopsy or other lesion removal, and a kit comprising the same. The tissue marker device may comprise a radiopaque marker; and an encasement that encapsulates the radiopaque marker. The encasement may include at least one portion configured, upon deployment into a biopsy cavity, to resist penetrating tissue surrounding the biopsy cavity.

The tissue marker device and the kit may be used in the method described above.

The present teachings overcome the above problem by providing a unique tissue marker device and kit comprising the same. Among the benefits of the present teachings is that the tissue marker device can be delivered through the biopsy marker deployment device described above, resist re-entry into an opening in a biopsy needle and/or a biopsy marker deployment device, resist being drawn away from or otherwise migrating from the biopsy site (e.g., a biopsy cavity), resisting penetrating tissue surrounding the biopsy site (e.g., a biopsy cavity), being capable of biodegrading and/or bioresorbing within a patient in need of a biopsy or other lesion removal, or any combination thereof.

The tissue marker device may include a longitudinal axis, and the encasement may entirely surround the radiopaque marker.

The tissue marker device may be elongated along the longitudinal axis, and the encasement may entirely surround the radiopaque marker.

The encasement may have a plurality of differing cross-sectional geometries and/or profiles taken transversely to the longitudinal axis of the encasement.

The encasement may have at least one first end portion, an intermediate portion, and at least one second end portion that is spaced apart from the at least one first end portion.

At least one of the at least one first end portion or the at least one second end portion may include a cross sectional profile that is different from a cross-sectional profile of the intermediate portion.

The at least one first end portion and/or the at least one second end portion of the encasement may include at least two discrete projections that extend away from the radiopaque marker.

The at least one first end portion and/or the at least one second end portion of the encasement may include at least two discrete projections that extend away from the radiopaque marker in a direction that is parallel with and/or diverge away from the longitudinal axis of the tissue marker device.

The at least one first end portion and/or the at least one second end portion of the encasement may include at least two discrete projections that extend away from the radiopaque marker in a direction that arcuately diverges away from the longitudinal axis of the tissue marker device.

The at least one first end portion and/or the at least one second end portion of the encasement may include at least two discrete projections that extend away from the radiopaque marker in a direction that arcuately diverges away from the longitudinal axis of the tissue marker device. The at least one first end portion and/or the at least one second end portion may have a geometry, before and/or after contacting a fluid in the biopsy cavity, which is sufficiently blunted to resist penetrating the tissue surrounding the biopsy cavity.

The encasement may be made from a biodegradable and/or bioresorbable material.

The encasement may be made from a biodegradable and/or bioresorbable material that, upon contact with the fluid within the biopsy cavity, swells and forms a blunted surface that resists penetrating the tissue surrounding the biopsy cavity.

The encasement may have at least two end portions and an intermediate portion therebetween. At least one or more of the at least two end portions may be multifurcated (preferably into a number of branches ranging from at least 2, and up to no more than 4, 8, 16, 25, 36, or even 49 branches) relative to the intermediate portion.

The encasement may have at least two end portions and an intermediate portion therebetween. At least one or more of the end portions may be multifurcated relative to the intermediate portion. One or more of the multifurcations may have an average cross-sectional area along their length that is less than two-thirds, preferable one-half, and specifically less than one-quarter, of the largest cross-sectional area of the tissue marker device.

The kit may comprise the tissue marker device and one or any combination of a biopsy device (preferably one configured and operable to remove lesions from and/or obtain tissue samples from a biopsy site of a breast or axillary lymph node, such as for diagnostic analysis of breast abnormalities, and to introduce the marker), a power cord, a power adapter, a single-use only biopsy probe for use with the biopsy device, a holder for the biopsy device, a biopsy needle, a biopsy marker deployment device, a driver, one or more medicaments, or any combination thereof.

The present disclosure contemplates the use of the tissue marker device, the biopsy probe assembly, the system, and the kit described herein, alone or in combination, with the method described herein.

The method with which the tissue marker device, the biopsy probe assembly, the system, and the kit may be used may comprise one or more of the following steps: a) providing a biopsy probe assembly including a probe body and a biopsy needle connected to the probe body, the biopsy needle including a longitudinal axis and an opening facing away from the longitudinal axis (optionally wherein the opening faces sideways relative to the longitudinal axis) and through which a biopsy sample can be taken; b) inserting the biopsy needle into a patient in need of a biopsy to position it at a biopsy site; c) removing a volume of tissue sample from the patient (optionally by employing a cutter coaxially disposed within the biopsy needle) while the biopsy needle is inserted into the patient at the biopsy site thereby defining a biopsy cavity; d) delivering the tissue marker device (optionally a radiopaque encapsulated marker) to the biopsy site through the opening with a biopsy marker deployment device (optionally the tissue marker device is prevented from puncturing the tissue around the biopsy cavity by one or more of: a largest dimension of the tissue marker device along its longitudinal axis, a length of the driver, one or more stops formed on the biopsy marker delivery device, and an angle of the ramp); and e) rotating or causing at least a portion of the biopsy marker deployment device to rotate (optionally manually, automatically with aid of a motor, or both) about its longitudinal axis (optionally within no more than 20 seconds, preferably 15 seconds, more preferably 10 seconds, still more preferably 5 seconds, or still more preferably 3 seconds following the tissue marker device (including any encasement) exiting the biopsy marker deployment device, the biopsy needle, or both) to bar re-entry of the marker into the biopsy needle through the opening; and f) optionally maintaining the biopsy needle at the biopsy site and rotating or causing rotation about the longitudinal axis of the biopsy needle at the time or of after when the step e) is performed.

The delivering step d) may include delivering the tissue marker device to a location at least partially, or fully, within the biopsy cavity using the biopsy marker deployment device and including a driver surrounded by a sheath having a longitudinal axis, and a step of rotating or causing to rotate the sheath about its longitudinal axis prior to the causing step e).

Radiographic detection of the tissue marker device may be performed following the deployment of the tissue marker device to confirm its presence no further than 10 mm, more preferably 7 mm, more preferably 5 mm from the biopsy cavity, optionally at a period of at least one year, more preferably at least two years, and still more preferably at least three years after the tissue marker device deployment. The causing step e) may include rotating the biopsy marker deployment device upon detecting (e.g., with a sensor) an exit of the tissue marker device (including any encasement) from the biopsy marker deployment device, the biopsy needle, or both. Optionally upon detecting an exit of the tissue marker device from the biopsy marker deployment device, the biopsy needle, or both, a signal may be transmitted to an electronic controller programmed to actuate a motor for causing the rotation of at least the sheath of the biopsy marker deployment device.

### BRIEF DESCRIPTION OF THE DRAWINGS

FIG. 1 is a side sectional view of a probe assembly according to the present teachings.

FIG. 2 is a side sectional view of a probe assembly according to the present teachings.

FIG. 3 is a side sectional view of a probe assembly according to the present teachings.

FIG. 4 is a side sectional view of a probe assembly according to the present teachings.

FIG. 5 is a side sectional view of a probe assembly according to the present teachings.

FIG. 6 is a side sectional view of a probe assembly according to the present teachings.

FIG. 7 is a plan view of a tissue marker device.

FIG. 8 is a plan view of a tissue marker device.

FIG. 9 is a plan view of a tissue marker device.

FIG. 10 is a plan view of a tissue marker device.

### DETAILED DESCRIPTION

As will be seen in further detail in the following discussion, in one aspect of the teachings, there is envisioned a method for introducing a marker into a biopsy site (e.g., a biopsy cavity). The method may comprise the steps of providing a biopsy probe assembly including a probe body and a biopsy needle connected to the probe body, the biopsy needle including a longitudinal axis and an opening facing away from the longitudinal axis (e.g., a side opening) and through which a tissue sample can be taken. The biopsy probe assembly may also include a cutter as described later herein. The method includes a step of inserting the biopsy needle into a patient in need of a biopsy to position it at a biopsy site. A step of removing a volume of a tissue sample from the patient, while the biopsy needle is inserted into the patient, at the biopsy site thereby defining a biopsy cavity can be performed (e.g., using the cutter as described herein, or otherwise using a cutter coaxially disposed within the biopsy needle). The method may include a step of delivering a marker (e.g., an encapsulated marker) to the biopsy site through the opening (e.g., through the side facing opening).

The step of delivering may employ a biopsy marker deployment device (e.g., one that is pre-loaded with one or more markers) having a longitudinal axis. The biopsy marker deployment device may be configured to be inserted within the needle (e.g., in common or parallel axial alignment with the needle). The biopsy marker deployment device may include a sheath that carries one or more markers that can be driven longitudinally along at least a portion of the sheath length for ejection through an opening. The opening may be a side facing opening which, in use during deployment, is brought into juxtaposed relation with the opening (e.g., side opening) of the needle.

The method may then include a step of rotating or causing the biopsy marker deployment device to rotate about its longitudinal axis to bar re-entry of the marker into the biopsy needle and/or the biopsy marker deployment device into and/or through the respective openings (e.g., side openings) of either or both the biopsy need and the biopsy marker deployment device. An additional step may follow that includes rotating the biopsy needle about its longitudinal axis.

More specifically, after a marker has exited the opening of the biopsy needle, the biopsy marker deployment device (e.g., the sheath of the biopsy marker deployment device) is rotated or caused to rotate about its longitudinal axis to bar re-entry of the marker into the biopsy probe assembly. For instance, the rotation is a sufficient amount (e.g., at least +/−120°, 150°, ideally 180° or 540° (or additional like increments (e.g., 270°, 360° or otherwise) of the previously recited amounts)), so that the opening (e.g., the side opening of the biopsy marker deployment device) faces away from the biopsy cavity. A wall portion of the biopsy marker deployment device (e.g., a wall portion of a sheath of the biopsy marker deployment device) may thus be in direct facing relation with the marker to prevent re-entry into and/or through the opening. As will be appreciated, the re-entry can be prevented as a result of the biopsy marker deployment device blocking a path toward re-entry.

After this, the biopsy needle likewise may be rotated about its longitudinal axis. For example, rotation may be at least +/−120°, 150°, ideally 180° or 540° (or additional like increments (e.g., 270°, 360° or otherwise) of the previously recited amounts). In this manner a wall portion of the biopsy needle may be in direct facing relation with the marker. The result of the rotation of the biopsy needle may be that a wall portion of the biopsy marker deployment device (e.g., a wall portion of a sheath of the biopsy marker deployment device) faces the opening of the biopsy needle, the opening of the biopsy marker deployment device faces an internal surface of a wall portion of the biopsy needle, or both.

It is contemplated that the biopsy needle may be rotated prior to rotation of the biopsy marker deployment device.

It is possible that the rotational orientations (relative to their longitudinal axes) of both the biopsy marker deployment device (e.g., its sheath) and the biopsy needle are such that a wall portion of each is in a blocking relation such that a deployed biopsy marker has no direct path toward the needle opening and the opening of the biopsy marker deployment device from which it was deployed.

More specific aspects of the above may include that the opening of either or both of the biopsy marker deployment device, or the needle, faces sideways relative to the longitudinal axis. The delivering step may include delivering the marker to a location at least partially, or fully, within the biopsy cavity. For example, the delivering step may include delivering the marker to a location at least partially, or fully, within the biopsy cavity using a biopsy marker deployment device including a driver surrounded by a sheath having a longitudinal axis and performing or causing to be performed a step of rotating the biopsy marker deployment device (e.g., at least its sheath) about its longitudinal axis upon or after the marker exits the biopsy marker deployment device. The delivering step may include maintaining the biopsy needle at the biopsy site during rotation about the longitudinal axis of the biopsy marker deployment device (e.g., to cause rotation of its sheath). The causing step may include rotating the biopsy marker deployment device manually, automatically with aid of a motor, or both. The causing step may include rotating the biopsy marker deployment device within no more than 20 seconds, preferably 15 seconds, more preferably 10 seconds, still more preferably 5 seconds, or still more preferably 3 seconds following the marker (including any encasement thereof) exiting the biopsy marker delivery device, the biopsy needle, or both.

The delivering step may include rotating the biopsy marker deployment device (i.e., at least its sheath) upon detecting (e.g., with a sensor) an exit of the marker (including any encasement thereof) from the biopsy marker delivery device, the biopsy needle, or both. For example, upon detecting an exit of a marker a signal may be transmitted to an electronic controller programmed to actuate a motor for causing the rotation of the biopsy marker deployment device. Alternatively, a signal may be transmitted to an electronic controller programmed to issue a visual, audible, or tactile indicator to a user to prompt the user to rotate the biopsy marker deployment device.

To confirm efficacy of the marker deployment and/or as part of a monitoring of the patient, radiographic detection of the marker may be performed following the deployment of the marker to confirm its presence no further than 10 mm, more preferably 7 mm, more preferably 5 mm from the biopsy cavity.

The teachings herein also contemplate a biopsy probe assembly configured for use in accordance with any of the above method steps. The biopsy probe assembly may be an assembly as described in more details herein. The assembly may include a probe body and a biopsy needle connected to the probe body, the biopsy needle including a longitudinal axis and an opening facing away (e.g., a side opening) from the longitudinal axis and through which a biopsy sample can be taken; a biopsy marker deployment device configured to advance one or more markers through the side opening of the biopsy needle; a motor that may be adapted to be automatically actuated, upon a marker exiting the opening of the biopsy needle, to rotate the biopsy marker deployment device (e.g., at least its sheath) about its longitudinal axis to bar re-entry of the marker into the biopsy needle through the opening; or any combination thereof. The motor may also be adapted to be automatically actuated upon or after a marker exits the opening to rotate the needle as well, before, simultaneously with, or following rotation of the biopsy marker deployment device (preferably following rotation of the biopsy marker deployment device).

A system may be employed to include the above-noted biopsy probe assembly, or any biopsy probe assembly described herein. The system may include an electronic controller in signaling communication with the motor, a sensor that detects an exit of a marker from the opening of the biopsy needle, or both. Upon the sensor detecting the exit, it may issue a sensor signal to the electronic controller. In response to the sensor signal the electronic controller may issue an actuation signal to the electric motor. The actuation signal may cause rotation of the biopsy marker deployment device (e.g., at least its sheath), the biopsy needle or both.

Turning, in more detail, to the respective components useful in accordance with the present teachings, generally applicable to all embodiments, it is envisioned that the teachings will employ a biopsy device. The biopsy device may include a biopsy probe assembly. The biopsy probe assembly can include a biopsy needle extending distally from a probe body. The biopsy needle may have a longitudinal axis. The biopsy needle may be connected in fixed position relative to the probe body, or it may be rotationally connected with the probe body (i.e., the biopsy needle may be rotated about its longitudinal axis while the probe body remains in a fixed position).

The biopsy needle may be dimensioned and configured to aid in taking one or more tissue samples, to facilitate insertion of a marker and/or some other matter into the patient, or both. The biopsy needle may include an opening through which a tissue sample can be taken, or matter may be introduced into a biopsy site. The matter may include a therapeutic agent, a marker, a lubricant, collagen, antibiotics, antiseptics, radiation seeds, chemotherapeutic agents, thrombosis activating agents, or any combinations thereof. For example, the biopsy needle may include a side opening. The side opening may be located at a distal end region of the biopsy needle. The biopsy needle may terminate at a distal tip portion. The distal tip portion may include a blade, a point, or both. The blade and/or point of the biopsy needle may enable it to cut tissue to access a site for retrieving a tissue sample. The biopsy needle may define a trocar.

A cutter moveable relative to the biopsy needle to sever tissue may be coupled with the probe body. The cutter may be located within the biopsy needle. The cutter may have a generally tubular configuration.

The cutter may lie coaxially within the biopsy needle at least partially along the length thereof. The cutter may have a longitudinal axis that is parallel with or coaxial with the longitudinal axis of the biopsy needle. The cutter, the biopsy needle, or both may be rotatable about their respective longitudinal axes relative to each other, relative to the probe body, or both.

In the delivering step, the marker may be prevented from puncturing the tissue around the biopsy cavity by one or more of: a largest dimension of the marker along its longitudinal axis, a length of the driver, one or more stops formed on the biopsy marker delivery device, and an angle of the ramp.

A biopsy marker deployment device (e.g., an elongated retractable deployment device) may be present. The biopsy marker deployment device may be configured to be used with the probe assembly to radiographically mark the location of the biopsy procedure (i.e., the biopsy site), such as a cavity defined by removal of a tissue sample. One or more markers may be preloaded within the biopsy marker deployment device prior to performance of a biopsy procedure (i.e., before the probe is inserted into a patient). Markers may be introduced within the biopsy marker deployment device at the time of the biopsy (i.e., during the procedure, but after the probe has been inserted into a patient). A plurality of markers may be carried in a common cartridge, from which they are ejected into the biopsy marker deployment device.

An example of a marker deployment device includes a driver (e.g., a rod) having a longitudinal axis and is dimensioned to fit within either or both of the cutter or biopsy needle. The driver is located within a sheath having a longitudinal axis and has a distal end that can contact a marker and, upon being advanced by a user longitudinally, push a marker to force the marker out of and through the opening (e.g., side opening) of the biopsy needle. The ejection may be aided by a deflection ramp located within the biopsy marker deployment device (e.g., within the distal tip portion of the sheath of the biopsy marker deployment device) proximate the distal tip of the biopsy needle and juxtaposed with the side opening. The present teachings contemplate that the ramp may be located within the needle, proximate to the distal tip thereof.

The driver may have a length such that when fully depressed or inserted relative to the sheath, the marker or any portion thereof is not pushed beyond the boundary of the biopsy cavity. In this regard, the marker may be prevented from puncturing the tissue surrounding the biopsy cavity. It has been discovered that in the event of biopsy cavity puncture, when a breast is released from compression, the marker may be carried deeper into the tissue from the region in the biopsy cavity which it punctures. This may be also applicable to other biopsy procedures involving compression of a region of the body. Further, regardless of compression, the marker may migrate, over time, deeper into the tissue from the region in the biopsy cavity which it punctures.

It is contemplated that the length of the driver may cooperate with one or more other features to prevent puncturing the biopsy cavity. The length of the driver and the largest dimension of the marker along its longitudinal axis may be configured to prevent puncturing the biopsy cavity. That is, the combined length of the fully depressed driver and the marker may not cause the marker to puncture the tissue. Moreover, the angle of the ramp may be configured to deflect the marker into a position that does not cause the marker to puncture the tissue. Moreover, the biopsy marker deployment device may comprise one or more stops that prevent further depression of the driver. One or any combination of the above features may be employed in the present teachings.

The stops may be located on the driver, the sheath, the finger grips, the plunger flange, or any combination thereof. The stops may be formed on at least two of these elements such that, upon depression of the driver, one or more stops of a first element eventually engages one or more stops of a second element. The stops may be in the form of projections extending along a transverse axis of the biopsy marker deployment device or at an angle therefrom. The stops may be formed on at least one of the aforementioned elements (e.g., the driver) such that, upon depression, the stops engage another element of the biopsy marker deployment device (e.g., the sheath).

One or more stops may cause a distal tip of the driver to be spaced about 1 mm or more, 2 mm or more, 3 mm or more, or even 4 mm or more from the distal tip of the biopsy needle when the driver is fully depressed relative to the biopsy needle. One or more stops may cause a distal tip of the driver to be spaced about 15 mm or less, 14 mm or less, 13 mm or less, 12 mm or less, 11 mm or less, or even 10 mm or less from the distal tip of the biopsy needle when the driver is fully depressed relative to the biopsy needle.

The length of the driver along the longitudinal axis thereof may be about 0.95 or less, 0.9 or less, 0.85 or less, or even 0.8 or less times the length of the biopsy needle along the longitudinal axis thereof. The length of the driver along the longitudinal axis thereof may be about 0.65 or more, 0.7 or more, or even 0.75 or more times the length of the biopsy needle along the longitudinal axis thereof.

As taught herein, a cutter may remove tissue from the biopsy site to form the biopsy cavity. The cutter may operate in the region around the opening formed in the biopsy needle to remove tissue. Thus, the biopsy cavity may be formed within the profile of the opening or even proximate thereto (e.g., no more than 2 cm, 1 cm, or even 0.5 cm from the profile defined by the opening). Thus, the combined length of the fully depressed driver, whether or not influenced by one or more stops, and the marker may prevent the marker from puncturing the tissue of the biopsy cavity defined by the aforementioned tissue removal.

The proximal portion of the biopsy marker deployment device may include a finger grip that surrounds the sheath or forms a part of the sheath. The finger grip is firmly connected to the sheath so that when it is rotated about a longitudinal axis of the biopsy marker deployment device, it causes the sheath to rotate. The drive rod is spring biased outwardly. A plunger flange is spaced from the finger grip portion and is depressible relative to the finger grip. For example, the proximal end portion of the drive rod may include a covering (e.g., an overmolded covering) over a portion of the drive rod length. The covering may have a diameter approximating the inner diameter of the finger grip portion or may be otherwise dimensioned and configured to be slidable within the finger grip portion. One or more detents, or other structure for realizing an interference fit, on one or both of the finger grip portion or the plunger flange portion helps resist dislodging of the plunger flange from the finger grip portion.

The biopsy probe assembly or any of its components may be configured for manipulation by a user, optionally with assistance of a device. The manipulation thus may be manually performed or at least partially automated. For example, the probe body or components thereof may be configured to include one or more components that are actuated, translated, rotated, driven, or otherwise manipulated by an assistance device (e.g., a mechanical, electrical, or electromechanical device).

It is possible that the probe body will be at least partially supported during use. For example, the probe body may be supported, in whole or in part, by a holster or holder. Either or both of the holster or holder may form part of a docking station. Either or both of the holster or holder, and optionally the docking station may be carried on a moveable cart. A probe guide device may be affixed to a surface and include a channel defining a wall or other surface against which the biopsy probe assembly can bear during operation. Optionally the probe guide device will help maintain a longitudinal orientation of the biopsy needle during operation.

The biopsy probe assembly may be directly or indirectly be coupled in operable relationship with a vacuum source (e.g., via a hose assembly) and a control module containing a programmable computer processor and a user interface (e.g., a touchscreen). The control module and vacuum source may be housed together within a housing. The control module and vacuum source may be housed together within a housing as a console. The control module and vacuum source may include a fan or blower for cooling motors, air vents, or other devices used for operating the control module and vacuum source. The entire assembly may be configured to draw power from a suitable power supply.

A vacuum source may be included in a system or apparatus of the present teachings. The vacuum source may be in fluid communication with the biopsy needle. Accordingly, it is possible to draw a reduced pressure through the biopsy needle and the cutter, thereby inducing a flow of matter (e.g., a tissue sample) away from a biopsy site. In this manner one or more tissue samples can be extracted with vacuum assistance. The control module may be suitably configured to provide power or issue signals, (e.g., wirelessly or wired) to drive a motor associated with a handpiece to operate the rotation of the needle, the rotation of the deployment device (e.g., its sheath) or both.

It is possible that the device used in accordance with the present teachings may be capable of retrieving multiple core biopsy samples from one insertion into breast tissue.

Turning now to a method of the teachings, it has been discovered that the previously discussed potential problem of marker migration can be obviated by a unique technique heretofore not recognized. In general, the method of the present teachings involves a step of providing an elongated retractable biopsy marker deployment device configured to drive a marker (e.g., an encapsulated marker) at least partially along a longitudinal axis of the deployment device and against a distal ramp for changing the direction of motion of the marker.

The method may include a step of providing a biopsy probe assembly. The biopsy probe assembly may be a biopsy probe assembly as described previously. Accordingly, this may include a step of providing a biopsy probe assembly that includes a biopsy needle that extends distally from a probe body, and that includes an opening (e.g., a side opening at a distal end region of the biopsy needle) through which a sample can be taken, or matter may be introduced into a biopsy site.

The method may include a step of providing a cutter. The step of providing the cutter may be part of the step of providing the probe assembly. Thus, the cutter may be provided with the biopsy probe assembly. The cutter may be as described previously. For instance, the cutter may have a tubular configuration and be operable for moving it (e.g., along a longitudinal axis of the biopsy needle) relative to the biopsy needle to sever tissue.

The method may include a step of providing a biopsy marker deployment device (e.g., an elongated retractable deployment device such as that described previously) configured to drive a marker (e.g., an encapsulated marker) at least partially along a longitudinal axis of the biopsy marker deployment device. The method may include a step of driving a marker against a distally located deflection ramp for changing the direction of motion of the marker.

The method may include a step of inserting or causing the biopsy marker deployment device to be coaxially inserted within the biopsy needle, the cutter or both. The method may include a step of inserting or causing the marker deployment device to be coaxially inserted within the biopsy needle, the cutter or both while the biopsy needle, the cutter or both are inserted within a patient in need of a biopsy. The method may include a step of positioning the biopsy marker deployment device so that that the opening of the biopsy needle (e.g., a side opening of the biopsy needle) is substantially juxtaposed with the deflection ramp of the biopsy marker deployment device.

The method may include a step of driving or causing the biopsy marker deployment device to drive the marker along the longitudinal axis against the ramp and through the opening (e.g., the distal side opening) of the biopsy probe assembly.

While keeping the biopsy probe assembly biopsy and/or the biopsy needle located at the predetermined location within a patient in need of a biopsy, after the marker has exited the opening of the biopsy needle, causing the biopsy marker deployment device (e.g., at least its sheath) to rotate about its longitudinal axis to bar re-entry of the marker into the biopsy needle.

As will be seen with respect to an example of an embodiment herein, it is also contemplated that the biopsy marker deployment device will include a sheath having a longitudinal axis that will also be rotated or caused to be rotated about its longitudinal axis in performance of the described method.

Thus, it will be seen, and it is generally contemplated that at the time of and/or immediately following ejection of the marker, a step of rotating the marker delivery device sheath will take place. The effect of that step and a simultaneous or subsequent rotation of the biopsy needle could be that at the time of ejection into the cavity, and preferably at the time of biopsy needle removal from a patient one or both the biopsy needle and the sheath of the biopsy marker deployment device are both in a rotated orientation (e.g., at least a 90°, 120°, 150° or 180° rotated orientation) relative to their respective orientations during ejection of the marker. In this manner it is envisioned that, immediately prior to withdrawal of the biopsy needle, at least the opening in the biopsy needle is facing away from the biopsy cavity. As described previously, it is possible that the ramp and opening of the biopsy marker deployment device (e.g., at least its sheath) also face away from the opening in the biopsy needle.

It should be understood that the description of method steps as including a step of “causing” also include a step of performing the underlying action that is “caused.” For example, a description of a step of “causing a rotation of a biopsy probe biopsy needle about its longitudinal axis” includes a description of “rotating a biopsy probe biopsy needle about its longitudinal axis.”

For all embodiments, the steps of causing as described herein may be performed any of a number of ways. By way of example, causing can be performed by instructing a user, an automated instrument or both to perform the step. Causing can be performed by providing audible verbal instructions, visual verbal instructions, audible signals (e.g., nonverbal sounds outputted through a speaker), visual signals (e.g., via a display device), tactile instructions (e.g., resistance to rotation, a vibrational output to a user handpiece, a foot pedal or the like), or any combination thereof.

In one example of the present teachings, the probe assembly may include suitable sensors for detecting the position of the various components relative to each other. For example, the biopsy probe assembly may include a sensor that detects when a marker has exited the opening of the probe assembly. In response to detection of the exit of the marker, the sensor may issue a signal that causes rotation of the biopsy needle, the biopsy marker deployment device (e.g., at least its sheath), or both about its longitudinal axis. For example, such a signal may be sent to an electronic controller that is programmed to actuate a motor to rotate the biopsy marker deployment device about its longitudinal axis. Such a signal may be sent to an electronic controller that is programmed to provide audible instructions, visual verbal instructions, audible signals (e.g., nonverbal sounds outputted through a speaker), visual signals (e.g., via a display device), tactile instructions (e.g., resistance to turning the canula, a vibrational output to a user handpiece, a foot pedal, or the like), or any combination thereof to cause a user to manually rotate the biopsy marker deployment device about its longitudinal axis.

In general, applicable to all embodiments, the step of causing a rotation of the biopsy marker deployment device (e.g., at least its sheath) about its longitudinal axis is performed less than 20 seconds, less than 10 seconds, less than 5 seconds, or less than 3 seconds after the biopsy marker deployment device has been depressed to eject the marker. The step of causing a rotation of the biopsy marker deployment device (e.g., at least its sheath) about its longitudinal axis may be performed before any residual compressive forces at the biopsy site can act to urge the marker back into the biopsy needle opening from which it was ejected.

Other aspects of the teachings, for all embodiments, may include employment of a sleeve over the biopsy needle that is translatable longitudinally along the biopsy needle for at least partially covering the side opening, and enabling a user to reduce the size of the opening.

For all embodiments, the methods, systems, and devices of the present teachings can be implemented using an automated biopsy device. Such an automated biopsy device may be vacuum assisted.

The methods, systems and devices of the present teachings can be implemented with an automated electronic control device that is programmed to automatically control the rotational orientation of the biopsy needle, and its associated opening (e.g., side opening), as well as the axial positioning and rotation of the cutter.

The methods, systems and devices of the present teachings can be employed with the teachings of U.S. Pat. Nos. 6,017,316 and 8,241,226, hereby expressly incorporated by reference in their entirety for all purposes. While examples described herein refer to biopsy samples from a breast, it should be understood that biopsy device may be used in a variety of other biopsy applications.

Markers for use herein may be suitably configured. They may be elongated. They may include one or more windings, a u-shaped configuration, a barbell configuration, a coil, a hook, a bowtie, a twist, opposing “c” or “u” elements, or otherwise. The markers may be at least partially encapsulated (e.g., by an elongated and/or cylindrical encasement). The markers may be any of the markers of U.S. Pat. Nos. 8,075,568; 8,075,569; and 8,454,629 hereby expressly incorporated by reference in their entirety for all purposes.

The markers may have a length greater than about 1 mm, 2 mm, or 3 mm. The markers may have a length less than 20 mm, 15 mm, or 10 mm. The markers may have a cross-sectional largest dimension (e.g., a cross-sectional diameter) of less than 7 mm, less than 5 mm, or less than 3 mm. The markers may have a cross-sectional largest dimension (e.g., a cross-sectional diameter) of more than 0.3 mm, or more than 1 mm.

The teachings herein also contemplate a unique tissue marker device. For example, the teachings envision a device that may include a radiopaque marker (e.g., as described above or otherwise); and an encasement that encapsulates the marker, wherein the encasement includes at least one portion configured, upon deployment into a biopsy cavity, to resist penetrating tissue surrounding the cavity. The device may have a longitudinal axis and may be configured so that an encasement entirely surrounds a marker. A device may be elongated along a longitudinal axis, and an encasement may entirely surround a marker.

An encasement may have a plurality of differing cross-sectional geometries taken transversely to a longitudinal axis of the encasement. An encasement has a plurality of differing cross-sectional profiles taken transversely to a longitudinal axis of the encasement.

An encasement may have at least one first end portion, an intermediate portion, and at least one second end portion that is spaced apart from the first end portion.

An encasement may have at least one first end portion, an intermediate portion, and at least one second end portion that is spaced apart from the first end portion, at least one of the first end portion or the second end portion includes a cross sectional profile that is different from a cross-sectional profile of the intermediate portion.

A radiopaque marker may be located entirely within an intermediate portion of the encasement. A portion of a radiopaque marker may extend into one or more end portions.

An end portion of an encasement may include at least two discrete projections that extend away from the marker.

It will be appreciated that, as applied to the various embodiments herein, a device may be elongated, such that it is longer than it is wide (e.g., for a generally cylindrical device, a length of the device may be longer than a diameter of the device measured at a transverse cross-section along a length of the device). An elongated device may have a longitudinal axis aligned in a direction of elongation.

A device may include an encasement having an end portion of the encasement that includes at least two discrete projections, also referred to herein as branches, that extend away from an encapsulated marker in a direction that is parallel with and/or diverges away from the longitudinal axis of the device.

An end portion of an encasement may include at least two discrete projections that extend away from the marker in a direction that arcuately diverges away from a longitudinal axis of the device.

An end portion of the encasement may include at least two discrete projections that extend away from the marker in a direction that arcuately diverges away from a longitudinal axis of the device and the end portion has a surface topology, before and/or after contacting a fluid in a biopsy cavity, which is sufficiently blunted to resist penetrating tissue surrounding the cavity. In this regard the surface topology may be characterized as being free of surfaces that define a barb, a spike, or other form of sharp and/or jagged edge or point.

An encasement may be made from a biodegradable and/or bioresorbable material. Bioresorbable refers to a material that upon placement within the human body starts to dissolve (resorbed) and slowly replaced by advancing tissue. Examples of materials may include tricalcium phosphate [Ca3(PO4)2], aliphatic polyesters such as poly(glycolic acid) (“PGA”), poly(lactic acid) (“PLA”), poly-DL-lactic acid (“PDLLA”), poly(caprolactone) (“PCL”), poly(trimethylene carbonate) (“PTMC”), poly(para-dioxanone) (“PPDO”), polylactic-polyglycolic acid copolymers, poly-L-lactic acid (“PLLA”), or any combination thereof. In comparison, the rate of hydrolysis of PLLA is found to be much slower than PGA. Other materials may include collagen, various polysaccharides such as cellulose, microbial polyesters, or any combination thereof. Other possible polymers may include one or more of poly(ortho esters), polyanhydrides, degradable polycarbonates, amino acid derived polymers in which conventional peptide bonds have been replaced by one or more other chemical linkages, or any combination thereof.

As seen, any of a variety of materials derived from lactide, glycolide, trimethylene carbonate, ρ-dioxanone, ε-caprolactone, or any combination thereof may be employed as an encasement material. This allows processing with common production technologies such as extrusion, injection molding, pultrusion, or otherwise.

It is contemplated that any combination of the above materials may be employed in fabricating the encasement.

An encasement may be made of any suitable material that is configured to possess a blunted surface topology prior to and/or following introduction of the encasement into a biopsy cavity.

An encasement may be made of any suitable material that is configured to possess a blunted surface topology prior to introduction of the encasement into a biopsy cavity, and shortly after introduction (e.g., less than 5 minutes, less than 3 minutes, or less than 1 minute) the blunted surface enlarges and/or becomes more blunted than its configuration before introduction. It is possible that an encasement may be made of any suitable material that is configured to possess a blunted surface topology prior to introduction of the encasement into a biopsy cavity, and to retain that blunted surface topology after introduction for at least 5 minutes, 10 minutes, 30 minutes, 1 hour, or possibly even longer.

An encasement may be employed from a material that, upon contact with a body fluid within a biopsy cavity swells, forms a gel, or otherwise changes form to result in formation of a blunted surface. The blunted surface may be essentially devoid of sharp edges or points, so that there is no topological feature that can penetrate tissue surrounding the biopsy cavity. In other words, the resultant blunting may improve resistance of the encasement to penetrating tissue surrounding the biopsy cavity as compared with an encasement having no blunted surface.

A blunted surface of an encasement may have a contoured (e.g., a continuously contoured) surface topology. In instances when an encasement has a contoured surface topology, it may include at least one curved surface that has one or more radii of curvature. It is possible that two or more surfaces having two or more radii of curvature directly and/or indirectly adjoin one another.

At least one or more of the end portions of an encasement may be multifurcated to define a plurality of branches that project away from the intermediate portion (preferably into a number of branches ranging from at least 2, and up to no more than 4, 8, 16, 25, 36, 49 branches) relative to an intermediate portion. It is possible that individual branches may have multiple sub-branches (e.g., in a fractal manner). It possible that two or more (or even all) branches extend from a common location. It possible that two or more (or even all) branches extend from a common location, and each have a length that varies relative to each other (as measured from the common location) that differs by less than 5 mm, 3 mm, or 1 mm. It possible that two or more (or even all) branches extend from a common location, and each have a length that varies relative to each other (as measured from the common location) that differs by at least 0.5 mm, 1 mm, or even 3 mm.

One or more ends portions of an encasement may have one or more radii of curvature relative to a longitudinal axis of an encasement. A distal tip of at least one branch of an end portion may have an outwardly curved surface relative to a longitudinal axis of an encasement, the surface having at least one radius of curvature. An end portion may contain a plurality of branches, and one or more of such branches may have a distal tip that may have an outwardly curved surface relative to a longitudinal axis of an encasement having at least one radius of curvature. It is possible that an encasement may include a plurality of surfaces as part of an end portion (e.g., at a distal tip of one or more branches). An encasement that includes a plurality of surfaces as part of an end portion may have a different radius of curvature for at least 2, 3, 4, or even more of the surfaces. For purposes of this description, the radius of curvature may be at a time within 3, 5, or even 10 minutes following contact with a body fluid in a biopsy cavity into which the encasement is introduced.

An end portion may have a maximum radius of curvature of at least two outermost surfaces that is at least 0.3 mm, 0.5 mm, or 1 mm. An end portion may have a maximum radius of curvature of at least two outermost surfaces that is less than 5 mm, 3 mm, or 2 mm. A surface may have two or more differing radii of curvature.

An encasement may have at least two end portions and an intermediate portion therebetween. At least one or more of the end portions may be multifurcated relative to an intermediate portion. One or more of the multifurcations may have an average cross-sectional area along its length that is less than two-thirds, (preferably one-half, and specifically less than one-quarter), of the largest cross-sectional area of the device.

Two or more (e.g., all) of branches defined in an end portion of an encasement may be parallel to each other. Two or more (e.g., all) of branches defined in an end portion of an encasement may be splayed at an angle (e.g., an acute angle, a right angle, and/or an obtuse angle) relative to each other. An end portion may include both of: i) two or more branches parallel to each other, and ii) two or more branches defined splayed at an angle (e.g., an acute angle, a right angle and/or an obtuse angle) relative to each other.

Before deployment (e.g., while located within a marker deployment device), the devices may be generally elongated and have a profile along their length to enable them to be expelled through the marker deployment device. The device may assume its intended shape following expulsion. This may arise, for example, through the use of its intrinsic material elasticity properties, through swelling as a result of water uptake, or both.

Without intending to be bound by theory, it is believed that the selective control over surface contour of the end portions of an encasement of a radiopaque marker device may contribute to preserving the location of the device in a patient (e.g., near or within a biopsy cavity proximate the biopsy site (e.g., proximate the walls of the biopsy cavity) so that the biopsy site may be readily located using conventional radiographic techniques to detect the marker of the device at least 3 months, 6 months, 9 months, 1 year, 2 years, 3 years, or even longer following the biopsy occurrence. For example, it is believed that use of the method and/or device described herein, following a biopsy and introduction of a marker into a patient, the marker may remain within 3 centimeters (cm), 2 cm, or even 1 cm of the biopsy site.

The teachings herein also contemplate a kit that contains components for performing a biopsy. For example, a kit may include any variation of the marker as has been described above; one or any combination of a biopsy device (e.g., a vacuum assisted biopsy device, such as a Mammotome® device), preferably one configured and operable to remove lesions from and/or obtain tissue samples from a biopsy site of a breast or axillary lymph node, such as for diagnostic analysis of breast abnormalities, and to introduce the marker; a power cord; a power adapter; a single-use only biopsy probe assembly for use with the biopsy device; a holder for the biopsy device; a biopsy needle; a biopsy marker deployment device; or any combination thereof.

It is also possible that the kits and/or the individual marker devices may include one or more medicaments. It is possible that an encasement is impregnated with or otherwise carries a medicament that promote hemostasis, reduce inflammation, or otherwise promote healing. Examples of medicaments include thrombin, tranexamic acid, an aminocaproic acid (e.g., epsilon-aminocaproic acid), ethamsylate, nafamostat mesylate, a vasopressin or an analogue thereof (e.g., a synthetic analogue, such as desmopressin or otherwise), or any combination thereof.

It is possible that a method as described herein may be employed to operate a biopsy device or system that is commercially available. For example, it is contemplated that a biopsy device or system available under the designation Mammotome® (e.g., the Mammotome Revolve™) may be used according to the method steps or may be modified to be controlled for use in accordance with the method steps herein. It is also possible that a method as described herein may be employed to operate a biopsy device or system as described in U.S. Pat. Nos. 8,075,568; 8,075,569; and 8,454,629 (all expressly incorporated by reference in their entirety for all purposes). Thus, it is possible that delivering the encapsulated marker to a biopsy site, wherein any step of delivering a marker to a biopsy site is free of a step of longitudinal repositioning of the biopsy needle or marker deployment device by the user in order that the encapsulated marker is precisely deployed within a biopsy site.

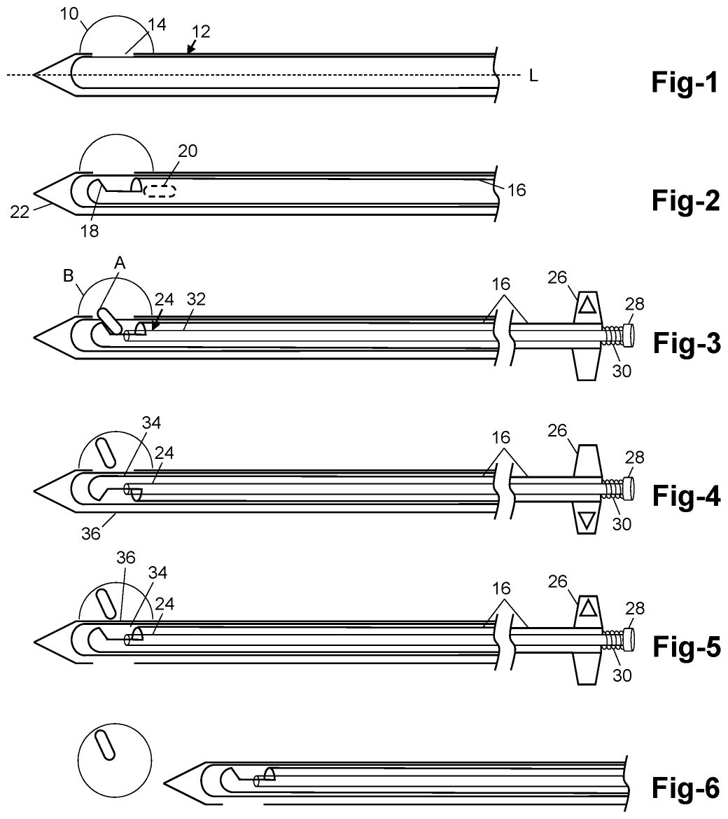

With reference to the drawings, one non-limiting illustrative example of the teachings is shown. FIG. 1 through FIG. 6 are side sectional schematics of an illustrative portion of a probe assembly for a biopsy device according to which, in a region adjoining a biopsy cavity 10, a biopsy needle 12 having a side opening 14 is shown, as seen in FIG. 1. A progression of steps is shown starting with locating a biopsy needle 12 at a biopsy site (FIG. 1) adjoining the biopsy cavity 10, inserting a biopsy marker deployment device 16 into the biopsy needle 12 (FIG. 2), ejecting a marker 20 through a side opening 14 of the biopsy needle 12 so it enters a biopsy cavity 10 (FIG. 3), a step of rotating the biopsy marker deployment device 16 during deployment (FIG. 4), a step of rotating the biopsy needle 12 after the marker 20 exits the opening 14 (FIG. 5), and a step of removing of the biopsy needle 12 (FIG. 6). The step depicted in FIG. 1 typically follows a step of cutting tissue using a cutter and removing the tissue (e.g., with vacuum assistance). The removal of the tissue has the effect of creating the biopsy cavity 10.

The drawings are not to scale, and the skilled person should recognize that portions of device are not shown in order to help avoid obscuring certain of the features.

While the biopsy needle 12 remains in position (e.g., at the location at which the cutter was used to take the sample) a biopsy marker deployment device 16 can be inserted into the biopsy needle 12. The illustrated biopsy marker deployment device 16 has a deflection ramp 18 to deflect the direction of a marker (e.g., an encased marker) 20 toward the side opening 14 of the biopsy needle 12. A blade 22 is depicted on the distal tip of the biopsy needle 12.

FIG. 3 shows the marker 20 being ejected through the side opening 14 of the biopsy needle 12, by a driver 24, so that the marker 20 enters a biopsy cavity 10. FIG. 3 also shows a finger grip 26 (having a position indicator, which may be in all embodiments) pointing upward. A plunger flange 28 for advancing the driver 24 is biased outward by a spring 30. The plunger flange 28 and finger grips 26 are spaced so a user can squeeze them toward each other to advance the driver 24. As described herein, a dimension of the marker 20, the length of the driver 24, the angle of the ramp 18, or any combination thereof may be configured to prevent the marker 20 from puncturing the tissue surrounding the biopsy cavity 10, such as a puncture caused, for example, by point A engaging or proceeding beyond point B.

FIG. 4 depicts a step of rotating the biopsy marker deployment device 16 during deployment. It can be rotated by way of the finger grip 26 which is connected with an outer sheath 32 that surrounds the driver 24 along a major portion of the length of the biopsy marker deployment device 16. Rotation of the finger grip 26 rotates the sheath 32. The rotation is shown as 180° (also the position if rotated 540° or additional increments of) 360° in this example. However, other rotation angles may be employed. Wall portion 34 of the biopsy marker deployment device 16 is shown facing the biopsy cavity 10.

FIG. 5 depicts a step of rotating the biopsy needle 12 after the marker 20 exits the side opening 14. As with the rotating of FIG. 4, it is illustrated as a rotation of 180° but can be a smaller or larger angle. Wall portion 36 is shown facing the biopsy cavity 10. It is seen that wall portions 34 and 36 together block a direct path of the deployed marker 20 back into the biopsy needle 12 and biopsy marker deployment device 16.

FIG. 6 shows a step of removing of the biopsy needle 12. The step of the removing of the biopsy needle 12 (for this and all embodiments) may be performed while the biopsy needle 12 is in its rotated orientation.

As seen in FIG. 5 and FIG. 6, for this embodiment (and in general for all embodiments), it is possible that the step of removal will take place while one or both the biopsy needle 12 and the sheath 32 of the biopsy marker deployment device 16 are both in a rotated orientation (e.g., at least a 90°, 120°, 150°, 180° or higher rotated orientation) relative to their respective orientations during ejection of the marker 20. In this manner it is envisioned that, immediately prior to withdrawal of the biopsy needle 12, at least the opening 14 in the biopsy needle 12 is facing away from the biopsy cavity 10. It is possible that the ramp 18 and an opening 38 of the biopsy marker deployment device 16 sheath 32 also face away from the opening 14 in the biopsy needle 12.

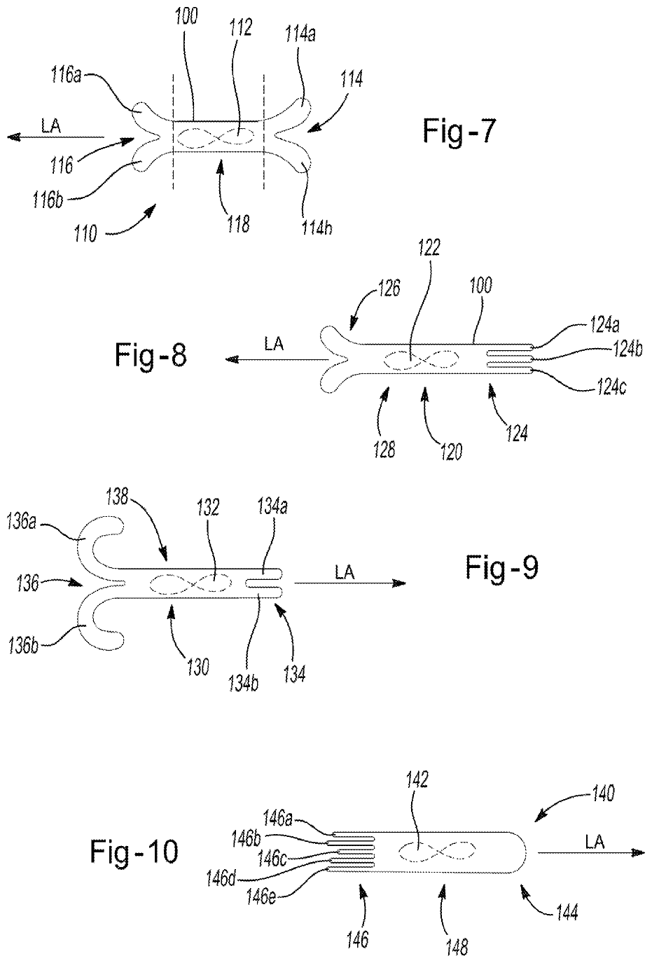

With attention now to the attached FIG. 7 through FIG. 10, examples of tissue marker devices of the present teachings are illustrated. Any of these examples, along with the tissue marker devices of the previous general teachings, are useful with the method previously described. Further, features depicted in one example may be employed in combination with features of other examples. The geometries shown may be the geometries assumed by the device after being deployed into a patient. For the embodiments illustrated, before deployment, the devices may be generally elongated and have a profile along their length to enable them to be expelled through a marker deployment device. The device may assume its intended shape following expulsion through the use of its intrinsic material elasticity properties, through swelling as a result of water uptake or both.

Markers of FIG. 7 through FIG. 10 are examples of markers that may be employed with the method illustrated in FIG. 1 through FIG. 6, though other markers can be employed as well, and the markers of FIG. 7 through FIG. 10 may be employed for other methods described herein.

FIG. 7 shows a tissue marker device 110. It includes a radiopaque marker 112 that is fully encapsulated in an encasement 100. The encasement 100 includes a first end portion 114 and a second end portion 116. An intermediate portion 118 connects the two end portions. In the depicted example (and for the other embodiments of the present teachings) the intermediate portion 118 may have a constant and/or varying cross-sectional shape (e.g., circular, polygonal, or otherwise) in a plane transverse to a longitudinal axis LA, which thereby defines a profile along the length (e.g., a cylindrical profile for a circular planar cross-section). Dotted lines show an example of a general location where the end portions 114, 116 may be joined with the intermediate portion 118. Other locations are possible as well (e.g., the marker may extend out of the intermediate portion 118 and into one or both end portions 114, 116).

As seen in FIG. 7, end portions 114, 116 may be split into branches. For example, a first end portion 114 is split into two diverging branches 114a and 114b. Likewise, the end portion 116 is split into two diverging branches 116a and 116b. The diverging branches are shown as being splayed relative to each other. The diverging branches shown in this example each have a generally constant profile over a major portion of their respective lengths. They terminate with a rounded tip, thereby affording a blunted surface topology for resisting undesired tissue penetration.

FIG. 8 illustrates another example, in which a tissue marker device 120 includes a radiopaque marker 122 fully encapsulated in an encasement 100. For this and other embodiments within the present teachings it is possible that some portion of the radiopaque marker 122 is not encapsulated by the encasement 100. In this example, a first end portion 124 includes a plurality of branches 124a, 124b, 124c, at least some of which are aligned parallel over most of their respective length with the longitudinal axis LA of the tissue marker device 120. In this example, it is also seen that a second end portion 126 is employed (depicted like that in FIG. 7, though any of the other configurations herein may be used). In contrast with the example of FIG. 7, this example depicts that the second end portion 126 differs in structure from the first end portion 124.

In general, as applicable to all of the embodiments of the present teachings, a second end portion may be the same as or it may differ in structure and/or size relative to that of a first end portion.

Returning again to FIG. 8, the resulting structure of the first end portion 124 may resemble a fringed or a frayed end of a fibrous cord. In this example, it is also seen that the branches 124a, 124b, 124c extend from different longitudinal positions relative to an intermediate portion 128.

In the examples of FIG. 7 and FIG. 8, an elongated structure having two opposing ends is shown. It is also possible that a device may have more than two end portions. For example, in FIG. 9, a tissue marker device 130 may include a marker 132 in an intermediate portion 138, and a portion 136 that is multifurcated so as to result in portions having splayed ends 136a and 136b that fold or roll back over themselves. The effect of this is to create what would appear to be three end portions, i.e., end portion 134, splayed end portion 136a, and splayed end portion 136b. In this embodiment, though any other structure can be employed in accordance with the general teachings (e.g., a structure such as in portion 136, with the end portions 136a and 136b), the end portion 134 includes multiple generally parallel projections 134a and 134b that are aligned with the longitudinal axis.

FIG. 10 illustrates a tissue marker device 140 encapsulating a radiopaque marker 142. A first end portion 144, and a second end portion 146 have an intermediate portion 148 located between them. In this example, the first end portion 144 is blunted (as seen by the continuously arcuate profile) and is not split into branches. The second end portion 146 is split into a number of branches. Each of the branches has a different length. Each of the branches 146a, 146b, 146c, 146d, and 146e are aligned with the longitudinal axis. It should be appreciated that two or more of the branches could be splayed relative to each other and need not be aligned with the longitudinal axis.

The present teachings contemplate that branches may be arranged about the longitudinal axis. By way of example, each of 2, and up to no more than 4, 8, 16, 25, 36, or even 49 branches may be arranged at 90, 180, 270, and 360 degrees (or any interval therebetween) about the longitudinal axis. The branches may be arranged symmetrically or asymmetrically relative to each other.

For all embodiments, it is possible that branches emanate from different longitudinal positions relative to a common transverse plane taken in an intermediate portion. For all embodiments, it is possible that branches have their distal tips terminate at different longitudinal positions relative to a common transverse plane taken in an intermediate portion. For example, though shown in FIG. 10 as emanating from (and terminating at) different transverse planes relative to longitudinal axis, some or all of the branches as emanating from (and terminating at) common transverse planes relative to longitudinal axis.

It is seen that in the depicted examples of FIG. 7 through FIG. 10 that distal tips all generally have rounded tips. It is possible in some instances that tips of one or more branches are configured to have a flat surface. In such instances, it is contemplated that the overall structure and/or material properties of any of the branches is such that they will deform (e.g., at least elastically and possibly plastically) promptly upon introduction into a patient so as to resist penetrating tissue. For example, ratio of length to width (or diameter) of a branch will obviate the need for blunting of that branch.

An intermediate portion of the encasement of tissue marker devices may have a constant transverse cross-sectional geometry along its length, and/or geometries that vary along the length. An intermediate portion of the encasement of the tissue marker devices may have a constant transverse cross-sectional area along its length, and/or areas that vary relative to each other along the length. By way of example, an encasement may have an overall configuration that is generally cylindrical, egg-shaped, cone-shaped, prism shaped, or a frustum-shape of any of the same.

For all embodiments, the biopsy probe, the system comprising the biopsy probe, the tissue marker device, or the kit described herein, or any combination thereof, may be employed with the method described herein.

For all embodiments, one or more elements of the biopsy probe, the system comprising the biopsy probe, the tissue marker device, or any combination thereof may be provided in the form of a kit.

For all embodiments, one or more elements of the biopsy probe, the tissue marker device, or the kit described herein, or any combination thereof, may be employed in the system described herein.

For all embodiments, the length of the opening of the biopsy needle and/or the biopsy marker deployment device (along its longitudinal axis) may be less than, generally equal to, or greater than the length of the marker and/or the encasement (including any branches thereof). Preferably the length of the opening of the biopsy needle and/or the biopsy marker deployment device (along its longitudinal axis) may be less than or generally equal to the length of the marker and/or encasement (including any branches thereof). Even more preferably the length of the opening of the biopsy needle and/or the biopsy marker deployment device (along its longitudinal axis) may be less than the length of the marker and/or encasement (including any branches thereof). In this regard, a first end of the marker and/or encasement (including any branches thereof) may exit the opening followed by a second end of the marker and/or encasement (including any branches thereof).

For all embodiments, the length of the marker and/or encasement (including any branches thereof) may be about 1 or more, 1.2 or more, 1.5 or more, 2 or more, or even 3 or more times the length of the opening of the biopsy needle and/or the biopsy marker deployment device (along its longitudinal axis).

For all embodiments, the width of the opening of the biopsy needle and/or the biopsy marker deployment device (along an axis transverse to its longitudinal axis) may be less than, generally equal to, or greater than the width (along an axis transverse to its longitudinal axis) of the marker and/or encasement (including any branches thereof). Preferably the width of the opening of the biopsy needle and/or the biopsy marker deployment device may be less than or generally equal to the width of the marker and/or encasement (including any branches thereof). More preferably, the width of the opening of the biopsy needle and/or the biopsy marker deployment device may be generally equal to the width of the marker and/or encasement (including any branches thereof). In this regard, the marker and/or encasement (including any branches thereof) may move along the ramp and into the cavity without canting in a generally left or right direction as it exits the opening.

For all embodiments, the ramp may be oriented at an angle of about 10 or more, 15 or more, 20 or more, 25 or more, 30 or more, 35 or more, 40 or more, or even 45 or more degrees relative to the longitudinal axis of the biopsy needle and/or the biopsy marker device. The ramp may be oriented at an angle of about 75 or less, 70 or less, 65 or less, 60 or less, or even 55 or less degrees relative to the longitudinal axis of the biopsy needle and/or the biopsy marker device.

Units depicted in the drawings are illustrative and not intended as limiting. They may vary as necessary for achieving the appropriate translation. Relative proportions depicted in the drawings are part of the teachings even if not expressly recited herein.

The disclosures of all articles and references, including patent applications and publications, are incorporated by reference in their entirety for all purposes.

The term “consisting essentially of” to describe a combination shall include the elements, components or steps identified, and such other elements, components, or steps that do not materially affect the basic and novel characteristics of the combination. For example, a recitation of “consisting essentially of” to describe a method for precise location of a marker as described herein, would include the identified steps; and could permit for such other steps that do not materially affect the ultimate location of the marker relative to a biopsy site. The use of the terms “comprising” or “including” to describe combinations of elements, components, or steps herein also contemplates embodiments that consist essentially of, or even consisting of, the elements, components, or steps.

Plural elements, components, or steps can be provided by a single integrated element, component, or step. Alternatively, a single integrated element, component, or step might be divided into separate plural elements, components, or steps. The disclosure of “a” or “one” to describe an element, component, or step is not intended to foreclose additional elements, components, or steps.

Unless otherwise stated, or derived from the specific context, references to “attach,” “attached,” “attaching” “attachment,” “connect,” “connected,” “connecting” “connection,” or conjugates of any of these and other synonymous terms include direct and indirect combinations.

Relative positional relationships of elements depicted in the drawings are part of the teachings herein, even if not verbally described. Further, geometries shown in the drawings (though not intended to be limiting) are also within the scope of the teachings, even if not verbally described.

Reference Numerals: 10 Biopsy cavity; 12 Biopsy needle; 14 Opening of biopsy needle; 16 Biopsy marker deployment device; 18 Ramp; 20 Marker; 22 Blade; 24 Driver; 26 Finger grips; 28 Plunger flange; 30 Spring; 32 Sheath; 34 Wall portion of biopsy marker deployment device; 36 Wall portion of biopsy needle; 38 Opening of biopsy marker deployment device; 100 Encasement; 110 Tissue marker device; 112 Radiopaque marker; 114 First end portion; 114a Branch; 114b Branch; 116 Second end portion; 116a Branch; 116b Branch; 118 Intermediate portion; 120 Tissue marker device; 122 Radiopaque marker; 124 First end portion; 124a Branch; 124b Branch; 124c Branch; 126 Second end portion; 128 Intermediate portion; 130 Tissue marker device; 132 Radiopaque marker; 134 End portion; 134a Projection; 134b Projection; 136 Portion; 136a Splayed end portion; 136b Splayed end portion; 138 Intermediate portion; 140 Tissue marker device; 142 Radiopaque marker; 144 First end portion; 146 Second end portion; 146a Branch; 146b Branch; 146c Branch; 146d Branch; 146e Branch; 148 Intermediate portion.

What is claimed is:

1. A tissue marker device configured for deployment into a patient in need of a biopsy or other lesion removal, the tissue marker device comprising: a) a radiopaque marker; and b) an encasement that encapsulates the radiopaque marker, wherein the encasement includes at least one portion configured, upon deployment into a biopsy cavity, to resist penetrating tissue surrounding the biopsy cavity.

2. The tissue marker device of claim 1, wherein the tissue marker device is defined by a longitudinal axis, the tissue marker device being elongated along the longitudinal axis, and wherein the encasement entirely surrounds the radiopaque marker.

3. (canceled)

4. The tissue marker device of claim 2, wherein the encasement has a plurality of differing cross-sectional geometries and/or a plurality of differing cross-sectional profiles taken transversely to the longitudinal axis of the encasement.

5-6. (canceled)

7. The tissue marker device of claim 4, wherein the encasement has at least one first end portion, an intermediate portion, and at least one second end portion that is spaced apart from the at least one first end portion, wherein at least one of the at least one first end portion or the at least one second end portion includes a cross sectional profile that is different from a cross-sectional profile of the intermediate portion.

8. (canceled)

9. The tissue marker device of claim 7, wherein the at least one first end portion and/or the at least one second end portion of the encasement includes at least two discrete projections that extend away from the radiopaque marker in a direction that is parallel with and/or diverge away from the longitudinal axis of the tissue marker device.

10. The tissue marker device of claim 7, wherein the at least one first end portion and/or the at least one second end portion of the encasement includes at least two discrete projections that extend away from the radiopaque marker in a direction that arcuately diverges away from the longitudinal axis of the tissue marker device.

11. The tissue marker device of claim 10, wherein the at least one first end portion and/or the at least one second end portion of the encasement includes at least two discrete projections that extend away from the radiopaque marker in a direction that arcuately diverges away from the longitudinal axis of the tissue marker device and the at least one first end portion and/or the at least one second end portion has a geometry, before and/or after contacting a fluid in the biopsy cavity, which is sufficiently blunted to resist penetrating the tissue surrounding the biopsy cavity.

12. (canceled)

13. The tissue marker device of claim 9, wherein the encasement is made from a biodegradable and/or bioresorbable material that, upon contact with the fluid within the biopsy cavity, swells and forms a blunted surface that resists penetrating the tissue surrounding the biopsy cavity.

14. (canceled)

15. The tissue marker device of claim 7, wherein the encasement has at least two end portions and an intermediate portion therebetween, at least one or more of the end portions are multifurcated, into between 2 and 49 branches, relative to the intermediate portion, and one or more of the multifurcations have an average cross-sectional area along their length that is less than two-thirds, preferable one-half, and specifically less than one-quarter, of the largest cross-sectional area of the tissue marker device.

16-20. (canceled)

21. A biopsy probe assembly including, a probe body and a biopsy needle connected to the probe body, the biopsy needle including a longitudinal axis and an opening facing away (e.g., sideways) from the longitudinal axis and through which a biopsy sample can be taken; and a marker deployment device configured to advance a tissue marker device through the side opening of the biopsy needle; and a motor that is adapted to be automatically actuated upon the tissue marker device exiting the opening of the biopsy needle to rotate the biopsy marker deployment device about its longitudinal axis to bar re-entry of the tissue marker device into the biopsy needle through the opening.

22. The biopsy probe assembly according to claim 21, wherein a length of the opening along the longitudinal axis is generally equal to or less than a length of the tissue marker device (including any encasement and/or branches thereof) along a longitudinal axis thereof; optionally wherein the length of the tissue marker device is about 1 or more, 1.2 or more, 1.5 or more, 2 or more, or even 3 or more times the length of the opening; and optionally wherein a width of the opening along an axis transverse to the longitudinal axis is generally equal to a width of the tissue marker device along an axis transverse to the longitudinal axis thereof.

23. The biopsy probe assembly according to claim 22, wherein the biopsy marker deployment device and/or the biopsy needle comprises a ramp; optionally wherein the ramp is within the distal tip portion (e.g., of a sheath) of the biopsy marker deployment device; optionally wherein the ramp is proximate a distal tip of the biopsy needle; optionally wherein the ramp is juxtaposed with the opening; optionally wherein the ramp is oriented at an angle of about 10 or more, 15 or more, 20 or more, 25 or more, 30 or more, 35 or more, 40 or more, or even 45 or more degrees relative to the longitudinal axis of the biopsy needle and/or the biopsy marker device; and optionally wherein the ramp is oriented at an angle of about 75 or less, 70 or less, 65 or less, 60 or less, or even 55 or less degrees relative to the longitudinal axis of the biopsy needle and/or the biopsy marker device.

24. The biopsy probe assembly according to claim 23, wherein a length of the driver along the longitudinal axis thereof is about 0.95 or less, 0.9 or less, 0.85 or less, or even 0.8 or less times the length of the biopsy needle along the longitudinal axis thereof; wherein the length of the driver along the longitudinal axis thereof is about 0.65 or more, 0.7 or more, or even 0.75 or more times the length of the biopsy needle along the longitudinal axis thereof.

25. The biopsy probe assembly according to claim 24, wherein the marker deployment device comprises one or more stops; wherein the one or more stops cause a distal tip of the driver to be spaced about 1 mm or more, 2 mm or more, 3 mm or more, or even 4 mm or more from the distal tip of the biopsy needle when the driver is fully depressed relative to the biopsy needle; wherein the one or more stops cause a distal tip of the driver to be spaced about 15 mm or less, 14 mm or less, 13 mm or less, 12 mm or less, 11 mm or less, or even 10 mm or less from the distal tip of the biopsy needle when the driver is fully depressed relative to the biopsy needle.

26. (canceled)

27. A method for introducing a tissue marker device into a biopsy cavity of a patient in need of a biopsy, comprising the steps of:
a) providing a biopsy probe assembly including a probe body and a biopsy needle connected to the probe body, the biopsy needle including a longitudinal axis and an opening facing away from the longitudinal axis and through which a biopsy sample can be taken;
b) inserting the biopsy needle into a patient to position it at a biopsy site;
c) removing a volume of tissue sample from the patient while the biopsy needle is inserted into the patient at the biopsy site thereby defining a biopsy cavity;
d) delivering the tissue marker device (optionally a radiopaque encapsulated marker) to the biopsy site through the opening with a biopsy marker deployment device; and
e) rotating or causing at least a portion of the biopsy marker deployment device to rotate about its longitudinal axis to bar re-entry of the tissue marker device into the biopsy needle through the opening.

28. The method of claim 27, wherein the opening faces sideways relative to the longitudinal axis.

29. The method of claim 28, further defined by one or more of the following:
the delivering step d) includes delivering the tissue marker device to a location at least partially, or fully, within the biopsy cavity using the biopsy marker deployment device and including a driver surrounded by a sheath having a longitudinal axis, and a step of rotating or causing to rotate the sheath about its longitudinal axis prior to the causing step e); optionally wherein the tissue marker device is delivered along a ramp of the biopsy marker deployment device;
a step f) that includes maintaining the biopsy needle at the biopsy site and rotating or causing rotation about the longitudinal axis of the biopsy needle at the time or of after when the step e) is performed;
the causing step e) includes rotating the biopsy marker deployment device manually, automatically with aid of a motor, or both, within no more than 20 seconds, preferably 15 seconds, more preferably 10 seconds, still preferably 5 seconds, or still more preferably 3 seconds following the tissue marker device (including any encasement) exiting the biopsy marker deployment device, the biopsy needle, or both;
the causing step e) includes the biopsy marker deployment device upon detecting (e.g. with a sensor) a of the tissue marker device (including an ent) from the biopsy marker deployment device, e biopsy needle, or both;
the removing step e) employs a cutter coaxially disposed within the biopsy needle; and
in the delivering step d), the tissue marker prevented from puncturing the tissue around the biopsy cavity by one or more of a largest dimension of the marker along its longitudinal axis, length of the driver one or more stops formed on the biopsy marker delivery device, and an angle of the ramp of or deployment device.

30. (canceled)

31. The method of claim 29, wherein radiographic detection of the tissue marker device performed following the deployment of the tissue marker device will confirm its presence no further than 10 mm, more preferably 7 mm, more preferably 5 mm from the biopsy cavity, optionally at a period of at least one year, more preferably at least two years, and still more preferably at least three years after the tissue marker device deployment.

32-34. (canceled)

35. The method of claim 31-34, wherein upon detecting an exit of the tissue marker device from the biopsy marker deployment device, the biopsy needle, or both, a signal is transmitted to an electronic controller programmed to actuate a motor for causing the rotation of at least the sheath of the biopsy marker deployment device.

36.-40. (canceled)
